(19) United States
(12) Patent Application Publication
(10) Pub. No.: US 2016/0095586 A1
(43) Pub. Date: Apr. 7, 2016

(54) SURGICAL IMPLANT SYSTEM AND METHOD

(71) Applicant: AMS RESEARCH CORPORATION, Minnetonka, MN (US)

(72) Inventors: John J. ALLEN, Mendota Heights, MN (US); Kevin R. ARNAL, Eden Prairie, MN (US)

(21) Appl. No.: 14/893,025

(22) PCT Filed: May 21, 2014
(86) PCT No.: PCT/US14/39018

§ 371 Date: Nov. 20, 2015

Related U.S. Application Data
(60) Provisional application No. 61/825,847, filed on May 21, 2013.

(51) Int. Cl. A61B 17/04; A61F 2/00

(52) U.S. Cl. CPC A61B 17/0401; A61F 2/0004; A61B 2017/00805

(57) ABSTRACT

An implant and anchoring system is provided. The implant can include a perineal anchor, and an obturator anchor or retropubic anchor. One or more sutures or suture loops can extend between and operatively connect the anchors. In certain embodiments, the anchors are introduced and deployed via a percutaneous transobturator procedure.

### PRIORITY

This Application claims priority to and the benefit of U.S. Provisional Patent Application No. 61/825,847, filed May 21, 2013, which is hereby fully incorporated herein by reference.

### FIELD OF THE INVENTION

The present invention relates to apparatus, tools and methods for treating pelvic conditions and, more particularly, systems and methods to support pelvic tissue by acting on, stabilizing, positioning or controlling the position of the perineal membrane or like anatomical structures.

### BACKGROUND OF THE INVENTION

It has been reported that over 13 million American men and women of all ages suffer from urinary and fecal incontinence. The social implications for an incontinent patient include loss of self-esteem, embarrassment, restriction of social and sexual activities, isolation, depression, and in some instances, dependence on caregivers. Incontinence is the most common reason for institutionalization of the elderly.

The urinary system consists of the kidneys, ureters, bladder and urethra. The bladder is a hollow, muscular, balloon-shaped sac that serves as a storage container for urine. The bladder is located behind the pubic bone and is protected by the pelvis. Ligaments hold the bladder in place and connect it to the pelvis and other tissue. The urethra is the tube that passes urine from the bladder out of the body. The narrow, internal opening of the urethra within the bladder is the bladder neck. In this region, the bladder's bundled muscular fibers transition into a sphincteric striated muscle called the internal sphincter. The urethra extends from the bladder neck to the end of the penis. The male urethra is composed of three portions: the prostatic, bulbar and pendulous portions. The prostatic portion is the widest part of the tube, which passes through the prostate gland. The rectum is the most distal portion of the gastrointestinal tract. The exterior opening of the rectum is the anus. Fecal continence is related to control of the exterior sphincter and interior sphincter of the anus.

Urinary incontinence may occur when the muscles of the urinary system are injured, malfunction or are weakened. Other factors, such as trauma to the urethral area, neurological injury, hormonal imbalance or medication side-effects, may also cause or contribute to incontinence. There are five basic types of incontinence: stress incontinence, urge incontinence, mixed incontinence, overflow incontinence, and functional incontinence. Stress urinary incontinence (SUI) is the involuntary loss of urine that occurs due to sudden increases in intra-abdominal pressure resulting from activities such as coughing, sneezing, lifting, straining, exercise and, in severe cases, even simply changing body position. Urge incontinence, also termed “hyperactive bladder,” “frequency/urgency syndrome,” or “irritable bladder,” occurs when an individual experiences the immediate need to urinate and loses bladder control before reaching the toilet. Mixed incontinence is the most common form of urinary incontinence. Inappropriate bladder contractions and weakened sphincter muscles usually cause this type of incontinence. Mixed incontinence is a combination of the symptoms for both stress and urge incontinence. Overflow incontinence is a constant dripping or leakage of urine caused by an overfilled bladder. Functional incontinence results when a person has difficulty moving from one place to another. It is generally caused by factors outside the lower urinary tract, such as deficits in physical function and/or cognitive function.

SUI is generally thought to be related to hypermobility of the bladder neck or an intrinsic urethral sphincter defect. A variety of treatment options are currently available to treat incontinence. Some of these treatment options include external devices, behavioral therapy (such as biofeedback, electrical stimulation, or Keel exercises), injectable materials, prosthetic devices and/or surgery. Depending on age, medical condition, and personal preference, surgical procedures can be used to completely restore continence.

Conservative management of SUI can include lifestyle changes, such as weight loss, smoking cessation, and modification of intake of diuretic fluids such as coffee and alcohol. Midurethral slings have been effective. One type of procedure, found to be an especially successful treatment option for SUI in both men and women, is a sling and support procedure.

A sling procedure is a surgical method involving the placement of a sling to stabilize or support the bladder neck or urethra. There are a variety of different sling procedures. Slings used for pubovaginal procedures differ in the type of material and anchoring methods. In some cases, the sling is placed under the bladder neck and secured via suspension structures or sutures to a point of attachment (e.g., tissue or bone) through an abdominal and/or vaginal incision. Examples of sling procedures are disclosed in U.S. Pat. Nos. 5,112,344; 5,611,515; 5,842,478; 5,860,425; 5,899,909; 6,039,686, 6,042,534, 6,110,101, 6,911,003, 6,652,450, and International PCT Publication No. 2008/057261, all of which are herein incorporated by reference in their entirety.

Fecal incontinence, like urinary incontinence, has proven to be challenging to treat. Patients whose fecal incontinence is caused by external anal sphincter injury is treated surgically, as with a sphincteroplasty. Other patients, though, are considered to have neurogenic or idiopathic fecal incontinence, and efforts to treat these patients has been less successful. Various procedures, such as postanal repair, total pelvic floor repair, muscle transposition techniques, dynamic graciloplasty, artificial sphincter procedures, and sacral nerve stimulation. Success has been limited, and the various treatment modalities can result in morbidity.

There is a desire for a minimally invasive yet highly effective treatment modality that can be used with minimal to no side effects for the treatment of both urinary and fecal incontinence. Such a modality should reduce the complexity of a treatment procedure, be biocompatible, should reduce pain, operative risks, infections and post operative hospital stays, and have a good duration of activity. Further, the method of treatment should also improve the quality of life for patients.

### SUMMARY OF THE INVENTION

The present invention can include surgical instruments, implantable articles, and methods for urological applications, particularly for the treatment of stress and/or urge urinary incontinence, fecal incontinence, and prolapse by implanting a paraurethral constraining device. The constraining device or implant can control and eliminate rotation of the urethra that is associated with incontinence.

Embodiments of the present invention can include apparatus and methods for treating urinary incontinence, fecal incontinence, prolapse, and other pelvic defects or dysfunctions, in both males and females using one or more implants to reinforce the supportive tissue of the urethra. The implants are configured to engage and pull (e.g., pull up) or reposition the supportive tissue, such as the perineal membrane. The perineal membrane is the fibrous membrane in the perineum that intersects the urethra and vagina near the midurethra location and can thus be stabilized or controlled in a manner that helps restore continence. As such, systems, methods and implants can be utilized to eliminate the need for mesh or other supportive structures under the urethra that is common with other incontinence slings. The implants can be shaped to facilitate such support, e.g., provided with anchoring end portions, barbs or other devices of many available shapes and configurations. One or more anchors or tissue engagement portions can be employed to attach and stabilize the implants or devices to tissue.

Embodiments of the present invention can provide smaller implants or devices, fewer implant or device components, thus reducing the size and number of incisions, improving implant manipulation and adjustment, the complexity of the insertion and deployment steps, and healing times.

The implants can limit or resist movement of tissue such as, for example, forward rotational movement of the urethra or surrounding tissue. The present implant embodiments can utilize a perineal incision or puncture and a paraurethral constraining device. Alternatively, the device may be implanted transvaginally.

In certain embodiments, one or more paraurethral support devices are provided. Paraurethral suspension elements are provided for the treatment of SUI and other disorders. The support, extension or suspension elements can apply mechanical traction to the urethra in a manner similar to a mini-sling device, wherein tension is applied at the midurethral position to lift and support that anatomical structure during stress events, such as coughing or physical activity.

An anchoring element or portion, can be fixed on each side of the urethra on the far side of a tissue layer that is known to have relatively high strength and toughness. Such anatomical structures can include the uterovaginal fascia, endopelvic fascia, perineal membrane or other anatomical features at which connective support of the urethra can be established.

A second anchor device, or engagement device, is placed in a lateral or superior position such that a connection between the medial and distal anchors (via a suture, mesh, wire or like connection) can provide tensile support for the urethra during stress events. The medial anchor device can be fixated to, or around, the tendinous arch of the levator ani (white line), the Cooper's ligament, the obturator foramen, obturator internus, abdominal fascia, sacrospinous ligament, prepubic fascia or muscle, the pubic symphysis cartilage, or other stable anatomical structures.

In various embodiments, the support of the perineal membrane can occur by setting an anchor or series of anchors in the perineal membrane via percutaneous transobturator access. A needle can be positioned in the leg muscle above the obturator and then advanced toward the urethra. When the resistance of the perineal membrane (or like tissue) is felt by the physician, the needle can be advance to pop or otherwise pass through the membrane. A finger near the urethra can be used as a guide to feel for the needle tip to ensure the urethra is not punctured or damaged, and to reposition the urethra relative to the advancing needle as needed. The anchor is deployed from the needle at the perineal membrane and the needle can be withdrawn, leaving the suture member trailing. The other opposing anchor can then be advanced into the leg puncture or like entry point along the trailing suture member and passed downward until it rests against the obturator muscle or membrane.

In lieu of the obturator tissue, support of the perineal membrane can be created by anchoring in the rectus muscle/ rectus fascia, coopers ligament, or a variety of other structures, fascia, muscles and ligaments accessible through passing tools through the tissue, through open, laparoscopic or robotically assisted laparoscopic methods.

### BRIEF DESCRIPTION OF THE DRAWINGS

FIG. 1 is a schematic view of various anatomical structures of the female pelvic region, including urinary and reproductive systems.

FIGS. 2-4 shows implant devices having a medial and distal anchors, in accordance with embodiments of the present invention.

FIGS. 5-6 show a percutaneous transobturator deployment method for an implant, in accordance with embodiments of the present invention.

FIGS. 7-8 show an implant with medial and distal anchors engaged with tissue via a transobturator deployment path, in accordance with embodiments of the present invention.

FIGS. 9-10 show various anchors, sutures and securement features for an implant, in accordance with embodiments of the present invention.

FIGS. 11-12 show an implant with medial and distal anchors engaged with tissue via a retropubic deployment path, in accordance with embodiments of the present invention.

### DETAILED DESCRIPTION OF PREFERRED EMBODIMENTS

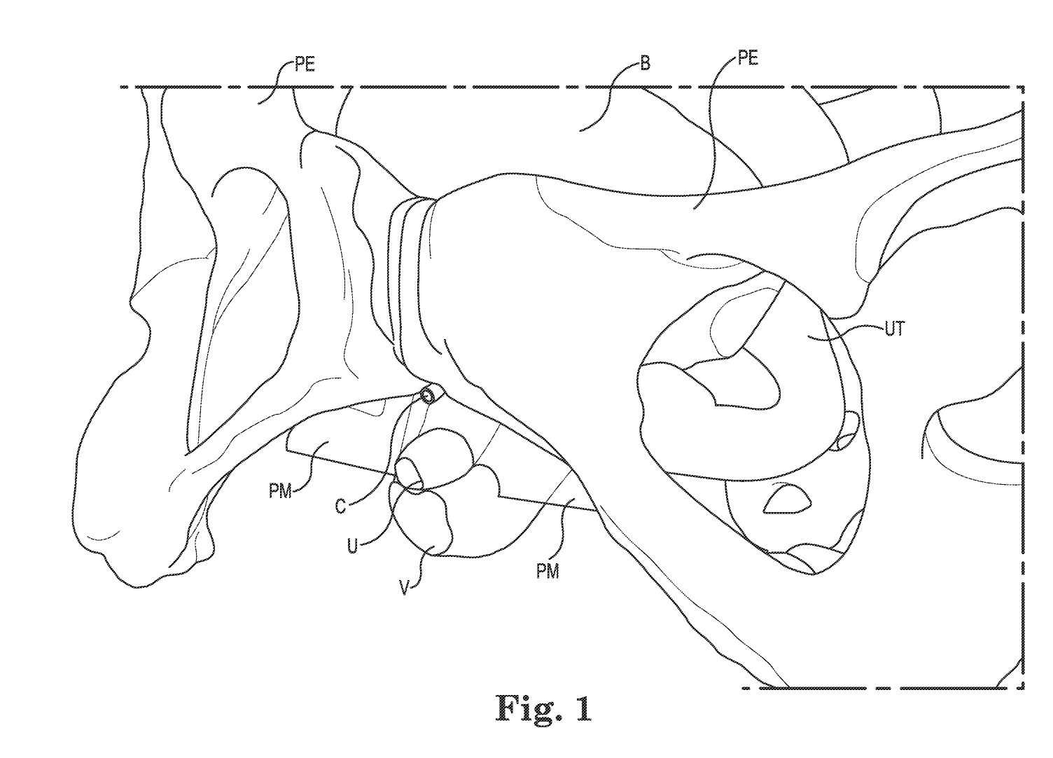

FIG. 1 shows a schematic view of relevant portions of the female pelvic region, and the urinary and reproductive system, including the pelvis PE, vagina V, uterus UT, urethra U, bladder B and the deep clitoral vein C. Further, a portion of the perineal membrane PM is shown at the midurethra/distal location, providing a viable paraurethral target for stabilizing or controlling the position and movement of the urethra to assist in restoring continence.

Embodiments of the present invention can include apparatus and methods for treating urinary incontinence, fecal incontinence, and other pelvic defects or dysfunctions, in both males and females using one or more lateral implants to reinforce the supportive tissue of the urethra. One or more implant devices 10 are configured to engage and pull (e.g., pull up) or reposition support tissue (e.g., paraurethral), such as the perineal membrane, uterovaginal fascia, endopelvic fascia, or other anatomical features at which connective support of the urethra can be established. The perineal membrane intersects the urethra and vagina at the midurethra/distal location and can thus be stabilized or controlled in a manner that helps restore continence. As such, the implants 10 can be utilized to eliminate the need for mesh or other supportive structures under the urethra that is common with other incontinence slings. The implants can be shaped to facilitate such support, e.g., provided with anchoring end portions 12, barbs or other devices of many available shapes, sizes and configurations, and one or more extension members 30. The one or more extension members 30 extend between one or more distal anchors 14 and one or more medial, or proximal, anchors 16.

Further, embodiments of the devices 10 and their corresponding anchors, suture loops, suture locks and/or tensioning mechanisms and techniques can be employed in a myriad of surgical procedures, including orthopedic, plastic surgery, cardiovascular, and like procedures to replace or supplement any traditional or other suture tightening and tensioning techniques. The devices and methods of the present invention can significantly reduce the time of surgical procedures by allowing for fast and efficient tensioning and locking securement of the implant without requiring a physician to tie sutures or introduce additional tensioning mechanisms.

Various embodiments of the extension members 30 can be constructed of a suture, a thin flat member, braided fibers, braided nano-fibers, an elongate mesh and other various materials and constructs. For those embodiments including braided nano-fibers, the extension member 30 can enhance and draw more collagen-producing cells to the material to promote tissue ingrown and healing.

The extension member 30 of certain embodiments of the present invention can be constructed to be generally flexible, or to have limited elasticity—e.g., bungee type attributes. For instance, the member 30 extending between the anchors 14 and anchors 16 can be an elongate member constructed of an elastomeric material having desirable tensile properties. As such, the member 30 can be stretched out for deployment and then released to provide desirable taut tension. The travel or stretching/rebound characteristics of the member 30 can vary depending on the particular elastomeric materials used in its construction. The extension member 30, such as a suture, can further include various extending tines or barbs to facilitate the tissue traction and grabbing during and after deployment.

The one or more opposing anchors 14, 16 or tissue engagement/securement portions can be employed to attach and stabilize the implants to the tissue, as well as provide selective adjustment. The anchors or engagement portions can be configured to engage soft tissue and can include various barbs, tines, serrated edges, extending fibers, or other similar structural feature to promote tissue fixation. The anchors can generally be small enough to be unnoticeable by both the patient and the patient's sexual partner. The anchors and other devices and components of the system 10 may be constructed from various biocompatible materials, such as known polymers and metals that promote long-term resilience, or other materials known to those skilled in the art.

In various embodiments, the one or more implants 10 can be placed in strategically located positions to pull up or otherwise tighten tissue and/or muscle. For instance, with certain embodiments, the tissue can be lateral or otherwise intersecting or attached (directly or indirectly) with the urethra to generally stabilize the anatomical structure of the patient. Various systems, devices, structures, techniques and methods, alone or in combination, as disclosed in U.S. Patent Publication Nos. 7,500,945, 7,407,480, 7,351,197, 7,347,812, 7,303,525, 7,025,063, 6,911,003, 6,691,711, 6,648,921, 6,612,977, 6,802,807, 2002/0161382, 2002/0147382, 2002/151762, 2004/0039453, 2008/0057261, 2008/0045782, 2010/0105979, 2011/0144417, and 2011/0201876, pending U.S. patent application No. 13/556,167, and International PCT Publication Nos. WO 2013/020134, WO 2013/016306, WO 2008/057261 and WO 2007/097994, can be employed with the present invention, with the above-identified disclosures being fully incorporated herein by reference in their entirety. The devices or structures described herein can be employed or introduced into the pelvic region of the patient percutaneously or in any other manner known by those of ordinary skill in the art.

Various embodiments are shown of the tissue constraining or positioning implant system 10 having one or more attachment points in one or more membranes or other target tissue locations. Embodiments can function to restrict, limit or control movement of the mid or distal urethra, or surrounding tissue. Further, embodiments can assist in resisting forward rotational movement of the urethra or surrounding tissue, and can provide support and tension during events, such as coughing or physical activity. Various advantages of the implant 10 embodiments depicted herein include, frontal access and simpler anatomy to address, less vascularity and bleeding, reduced risk of creating retention and de novo urge, and the ability to test for continence before surgery. Additionally, the implants 10 act to oppose rotational movement of the urethra, thereby eliminating or lessening the effects of stress urinary incontinence.

Certain of the devices 12, e.g., the proximal or medial anchor 16, can be generally provided in a back-to-back serial configuration, with a suture or like extension member extending to provide adjustable support between the anchor devices 14, 16. As shown in Figures, the anchor devices can include one or more first distal anchor devices 14, and one or more second medial anchor devices 16.

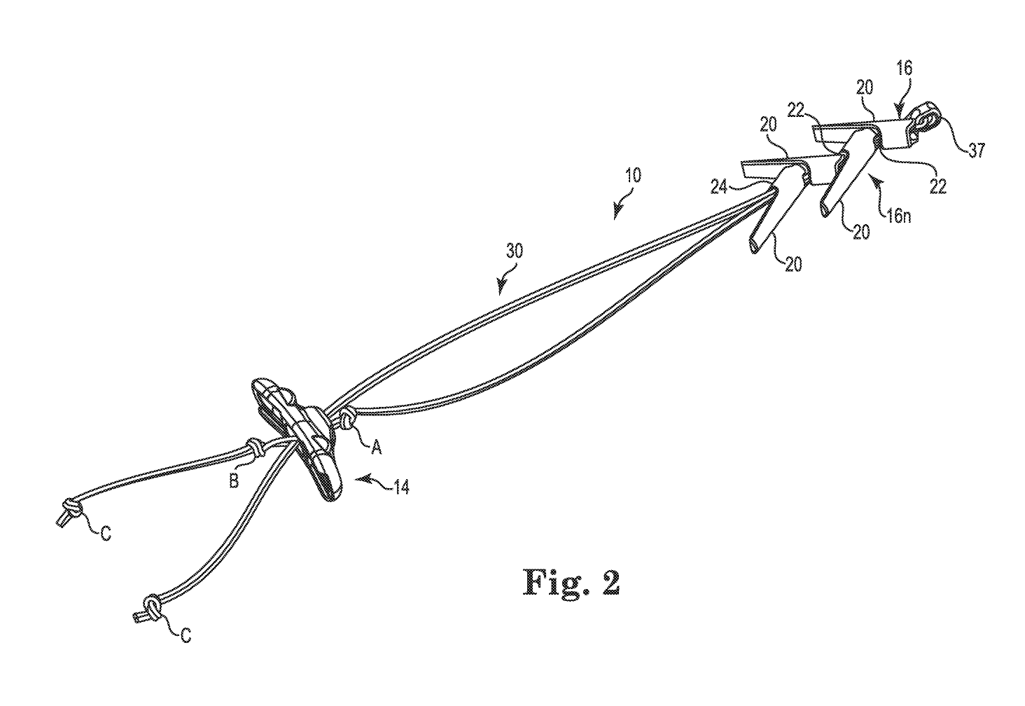

Referring generally to FIG. 2, the medial anchor devices 16 can include a body portion having one or more expandable barbs 20, a thru-aperture 22, and an opposing end 24. A suture, such as a suture loop 30, or like member, is adapted to string or thread through the respective apertures 22 of a series or array 16n of such anchors to define the general elongate and expandable configuration shown. The array of anchors 16n can be inserted within and along the interior lumen of a needle, cannula or like inserter or delivery tool.

Unlike conventional pelvic sling and implantation methods, the path from the perineal membrane to the medial anchor 14 of the present invention can follow a generally straight line into the obturator internus muscle, or like distal tissue. Furthermore, because it intersects the muscle at an oblique angle, more tissue can be engaged for securement.

In certain embodiments, the anchors 16 can be fabricated using an injection molding process, or from a molded resin material (e.g., 720FC resin, polycarbonate, PEEK, nylon), with an exemplary Polypropylene monofilament, or braided, suture, or suture loop 30, threaded therethrough. The components can be easily inserted through the needle and arranged in an alternating pattern—e.g., angular orientation pattern—along the suture 30. Of course, a myriad of alternate angular patterns and orientations are envisioned for embodiments of the invention depending on the particular deployment, anchoring and engagement needs. The suture 30 can be lightly tensioned to bring all the anchors 16 in the array 16n together while holding the pusher in place. The anchors 16 can include self-expanding structures or materials such that the anchors 16, or anchor array 16n, can be generally collapsed or reduced in sized during deployment, with or without a needle device, and expanded after penetration in the target tissue site to provide desired tissue engagement. Certain anchors 16 can include one or more shape memory portions, or living hinges, to facilitate this structural self-expansion upon deployment and tissue engagement. Further, embodiments of the medial anchor 16, or anchor array 16n, can include helical portions, threaded portions, hooks, clips, flexible barbs, textured surfaces, and like members or structures to promote tissue engagement. In addition, still other embodiments of the lateral anchor 16 can be adapted to include a plurality of anchors, extending from one or more separate members 30, spread out into multiple anchoring features for deployment into tissue to provide support and treatment. Such a spanning multi-anchor device 16 can create a neo-ligament to reduce or eliminate rotation of the urethra U or surrounding tissue through the use of multiple anchoring spots. The implant, anchoring and deployment devices, components, tools, techniques and methods disclosed in U.S. Patent Publication No. 2013/0023724 can be employed with, in whole or in part, embodiments of the present invention and, as a result, this patent publication is hereby fully incorporated herein by reference.

The systems, devices, configurations and methods disclosed herein have generally described anchors that are symmetrically, bilaterally, positioned above, below or on the side of the urethra. However, a single side deployment configuration can still achieve continence and is available with various embodiments. For instance, a single distal anchor 14 and medial anchor 16, or anchor array 16n, can be connected by one or more sutures to support and adjust the perineal membrane, above, below, or on a side of the urethra.

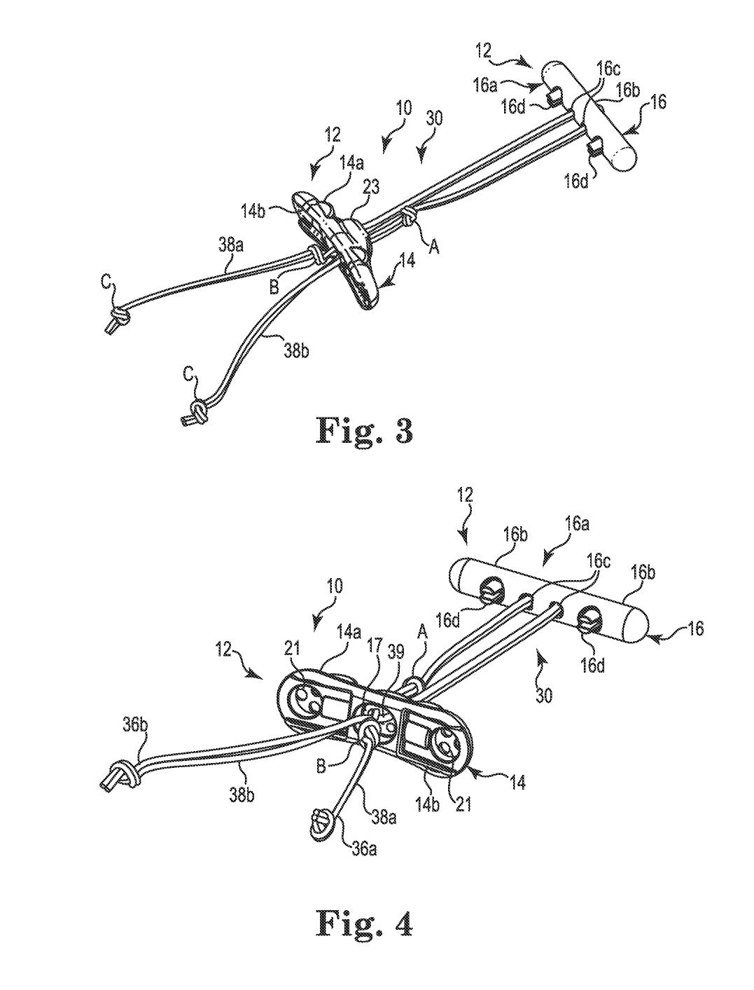

Referring again generally to FIGS. 3-4, the medial anchor device 16 can be a medial toggle anchor 16a having a body portion 16b, one or more loop apertures 16c, and one or more extending members 16d, as disclosed in International PCT Application No. PCT/U.S.14/30894, which is incorporated fully herein by reference. The toggle anchor 16a is generally elongate and adapted to be carried by a portion of delivery device (e.g., distal needle end 110) as further detailed herein, for anchoring within soft tissue, e.g., the perineal membrane. The anchor 16a can be constructed of a polymer material, such as PEEK or like materials, with the one or more loop apertures 16c configured to receive one or more suture members 30 such that the suture 38b enters a first aperture 16c, loops around a portion of the anchor 16a, and loops back into and through a second aperture 16c for a return path to the distal anchor 14. The one or more extending members 16d are also adapted to be carried within the delivery device (e.g., 100), and can facilitate alignment and prevent rotation of the anchor 16a while it is being carried by and deployed from the anchor 16a. Extending members 16d can also be adapted to fit within a slot on the delivery device such that anchor 16a is retained within the delivery device, and to provide stability of anchor 16a within soft tissue—e.g., acting as small barbs to increase its engagement with tissue.

The one or more distal anchors 14 are generally elongate and can extend out generally transverse from the connected suture loop 30. The anchor 14 can include a top side or surface 14a, a bottom side or surface 14b, one or more through-apertures. The through-apertures can be provided generally central to the medial anchor 14 and can be included within a cavity or pocket 17. A domed portion 23, or other like construct, can extend out from the top surface 14b, and can include the through-apertures and/or the pocket 17. The pocket 17 can be sized and shaped to partially or completely contain the knot B when the medial loop 30 is tensioned. In addition, the pocket 17 can serve to conceal the knot B such that it causes less irritation after implantation. Further, one or more additional apertures 21 can be provided with the medial anchor 14 to facilitate further attachment, and/or to provide openings to promote tissue in-growth.

In certain embodiments of the anchor 14, the one or more second through apertures includes a single aperture adapted to receive one or more sutures. The aperture can take on various sizes and shapes to facilitate the introduction and traversal of sutures or like members. In other embodiments, the one or more second through apertures includes two distinct and separate apertures also capable of taking on various shapes and sizes to accommodate sutures or like members. The inclusion of two apertures can provide better control of suture bending to promote self-locking behavior. The one or anchors 14 of the system 10 can be constructed of various metal or polymer materials including, for instance, PEEK or polypropylene.

FIGS. 2-4 show embodiments in which a suture loop 30 extends all the way to the anchor 16. With such a construct, the length between the anchor 14 and the anchor 16 is entirely available for adjustment. Alternatively, a pulley or pulley surface can be included with the anchor 16 itself (e.g., at the distal end of the implant), eliminating the need for a separate exterior pulley block at a portion of the length of the loop 30.

A loosening or lengthening length 38a and a cinching or tensioning length 38b of suture are provided below the underside 14b of the distal anchor 14. By passing the length 38b between the pair of sutures of the length 38a, the locking mechanism becomes self-locking upon actuation or tensioning between the anchors 14, 16 and requires no additional steps except to trim the suture length upon completion of the procedure. The locking occurs due to cinching from the suture 30, in conjunction with fact that the length 38b is forced into a bent configuration, at a relatively sharp angle, to generally prevent sliding or movement of the suture length 38a. Those embodiments having two distinct apertures in the anchor 14 can result in sharper bends 39 to the suture passing through them and, consequently, better locking behavior and securement. Namely, each suture of the pair bends directly over its own aperture edge surface to promote locking. Even with little or no tension on the anchor 14 or the respective suture portions, the suture does not slip easily or lengthen the loop. Moreover, this arrangement can be easily loosened or tightened by pulling on the appropriate end (e.g., 38b for tightening, 38a for loosening) and is not generally affected by whether the suture loop has been previously locked down.

The knots A, B are provided along the suture length and can be provided relatively close to one another to avoid backlash, yet far enough away to allow free movement of the adjustment end when desired. The functions served by knots A or B can alternatively be provided by other means, e.g., fusing or melting the suture at the same locations, applying a fixed clamping ring, overmolding a bead, et. One or more knots C, or like end or stop features, can be provided at or approximate the ends 36a, 36b of the suture loop 30 that are now provided below the anchor 14. The end features can include polymer or like colored tabs (e.g., yellow and blue) provided to assist in identifying the respective suture lengths and grabbing to pull or adjust for loosening and/or tensioning. In other embodiments, one or both of the ends 36b can include a loop formed from a reversal of the suture at that point, or an additional closed loop beyond one or both of the knots C or end features. Such a feature can serve as a ‘finger loop’ and can be used by the physician to make adjustments and to facilitate manual manipulation of the suture lengths.

As an alternative to knots, other devices, mechanisms or techniques can be employed to facilitate the cinching or locking of the device 10. For example, clamping rings (e.g., swaged rings), adhesive bonds, fusion (melting) of the suture material, or using a specifically designed part that has a split midline (in place of the two sutures between the knots A, B) can be used to facilitate the tensioning or locking features of the present invention. As described herein, the bend portion of the loop can serve as a pulley or pulley surface when engaged with the loop 30. Because of the limited amount of movement that may be required during a one-time or limited adjustment of the implant, and the described suture materials, there is not need for a long lasting bearing surface or separate pulley device in certain embodiments.

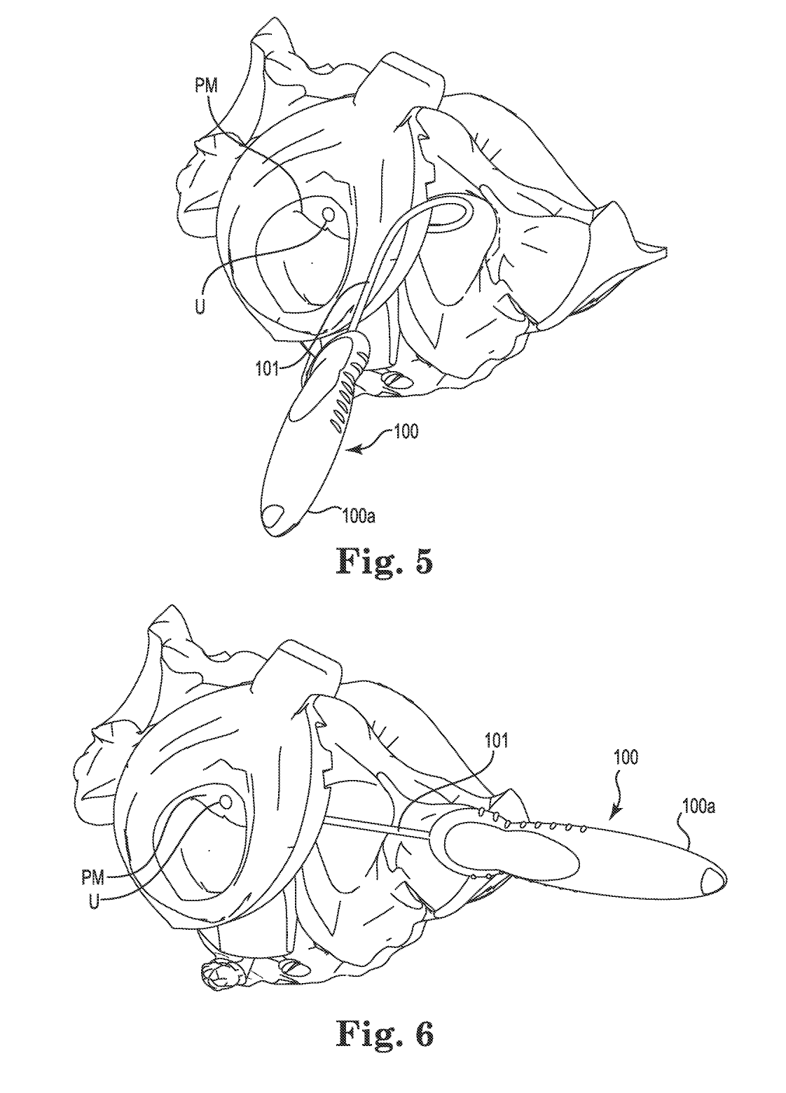

The support of the perineal membrane PM can occur by setting an anchor or series of anchors in the perineal membrane PM via percutaneous transobturator access, as shown in FIGS. 5-8. By using a needle device 100, e.g., going through the leg muscle, through the obturator, and to the perineal membrane, one or more distal anchors 14 can be placed in the perineal membrane PM. The device 100 can include a handle 100a, and a needle 101. The anchors can be deployed, and with a trailing member, e.g., a suture or like member 30, attached to the anchor 16, a separate distal anchor 14 can be deployed in or against the obturator muscle, or membrane, with the opposing anchors 14, 16 tensioned relative to each other to support the perineal membrane using the trailing member. A similar support can be provided on the contralateral side of the urethra U if needed to create continence.

A tip of the needle 101 can be positioned in the leg muscle above the obturator O and then advanced toward the urethra U (FIGS. 5-6). When the resistance of the perineal membrane PM (or like tissue) is felt by the physician, the needle 101 can be advanced to pop or otherwise pass through the membrane. A finger near the urethra U can be used as a guide to feel for the needle tip to ensure the urethra U is not punctured or damaged, and to reposition the urethra U relative to the advancing needle 101 as needed. The anchor 16 is deployed from the needle 101 and the needle can be withdrawn, leaving the suture member 30 trailing. The anchor 14 can then be advanced into the leg puncture or like entry point along the trailing suture member 30 and passed downward until it rests against the obturator O muscle or membrane.

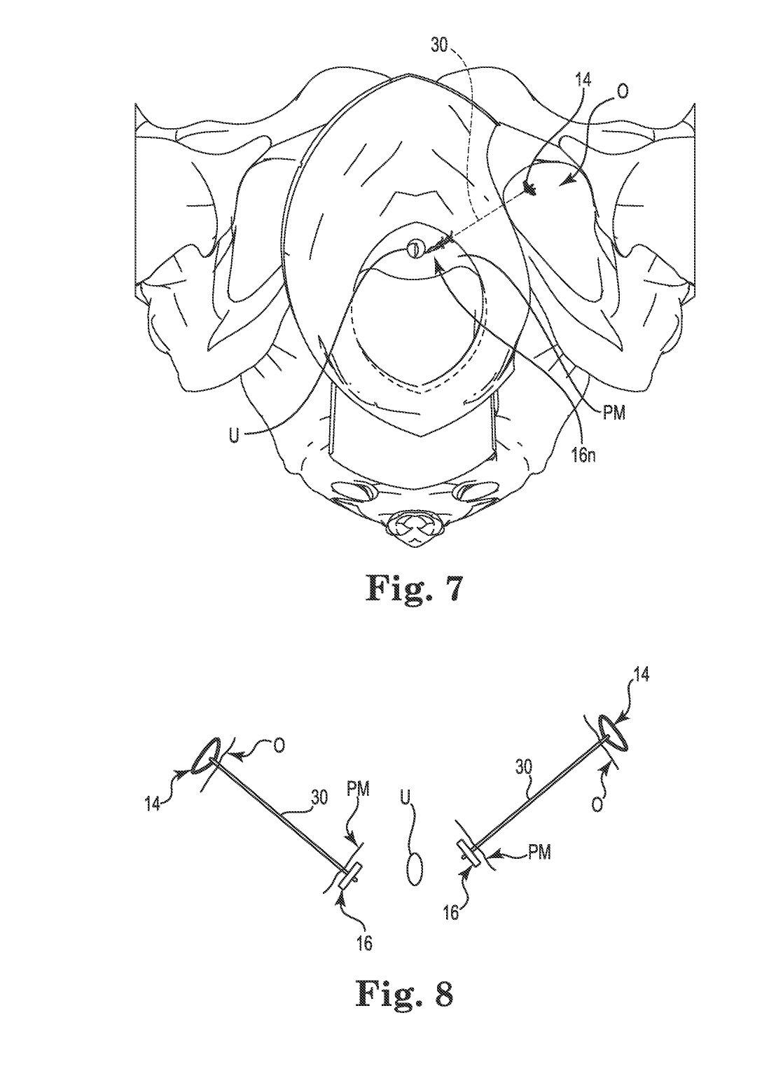

As shown in FIGS. 7-8, tensioning of the implant 10 can be achieved by pulling on the suture member 30, as disclosed herein, thereby using the anchor 14 at the obturator O as an anchor or leverage point, creating tension on the perineal membrane at the perineal anchor 16 to adjust support. This procedure can be carried out on the contralateral side as needed.

A curved needle 101 can be advantageous for advancing from the introductions to the perineal membrane PM. In certain embodiments, the needle 101 can be solid, or a generally hollow cannula.

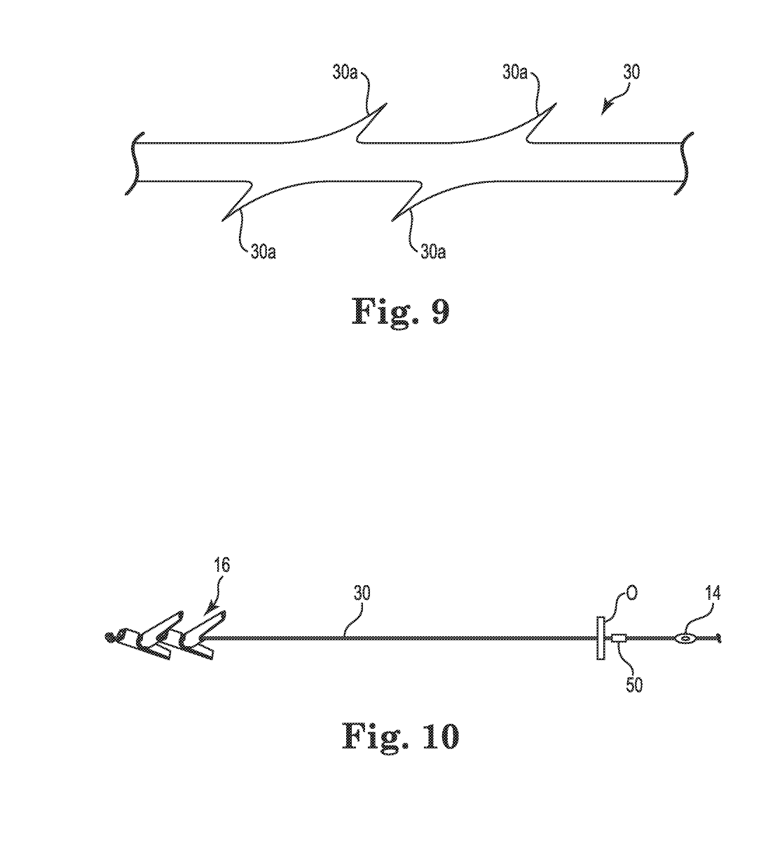

While various structures are depicted for anchors 14, 16, any of the anchors or tissue fixation devices and methods described herein can be employed for either of the anchors. In certain embodiments, sutures can be used instead of anchors. For instance, a quilled suture, having surface textures, tines or protrusions 30a, can be used for suture 30 and in place of having one of more of the anchors 14, 16 to facilitate fixation, tensioning and adjustment, as shown in FIG. 9. Other embodiments, such as those disclosed in FIG. 10, can include a perineal membrane anchor 16, a suture 30, and a suture knot or one-way stopper 50. The stopper 50 or knot can take the place of, or can be used in conjunction with, the opposing or proximate anchors.

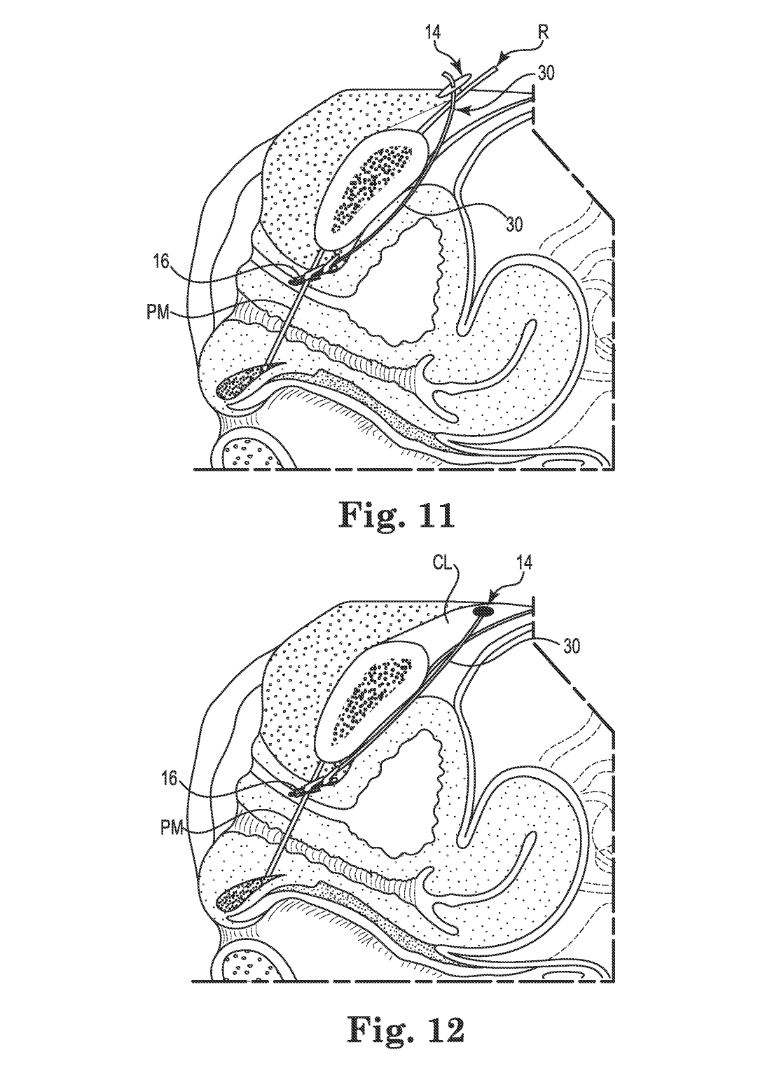

As depicted in FIGS. 11-12, in lieu of the obturator tissue O, support of the perineal membrane PM can be created by anchoring (e.g., anchor 14) in the rectus muscle/rectus fascia, cooper's ligament, or a variety of other structures, fascia, muscles and ligaments accessible by passing tools through the tissue, through open, laparoscopic or robotically-assisted laparoscopic methods. By placing anchors in these locations and through these methods, a suture 30 or other suitable connecting or trailing member can be passed from the one or more anchor 14 to the perineal membrane PM which can be supported through the use of the anchor 16, or suture knot techniques. These methods are employable in procedures for both men and women.

The one or more anchors 14 can be placed in the rectus R muscle, fascia or like tissue, and the one or more anchors 16 can be placed in the perineal membrane PM, as shown in FIG. 11. The anchors 14, 16 can be tensioned relative to one another via a tensioning mechanism, or by tying knots at either or both anchors 14, 16. The anchors 14, 16 can be introduced and deployed with the needle device 100, passing from the urethra to the rectus muscle or fascia R, or from the rectus muscle or fascia down to the perineal membrane PM—e.g., via a bottom-up or top-down approach. In one approach, the needle 101 is passed lateral to the bladder or urethra while hugging or following the posterior surface of the pelvic bone, and then placing the implant 10 and anchors. With such an approach, the needles can be solid or hollow. The anchors 14, 16 can be positioned internal or external to the needle during introduction and deployment. The suture 30 joining the anchors 14, 16 can be tensioned to reinforce the perineal membrane PM using a variety of mechanisms, such as the locks, loops and like mechanisms and techniques described herein.

Another embodiment of the procedure to introduce and implant and support the perineal membrane PM is through an open, laparoscopic or robotically assisted laparoscopic procedure, utilizing Cooper's ligament CL as an anchor location (e.g., with anchor 14)—supporting the perineal membrane PM laterally or bilaterally using the techniques disclosed herein, as shown in FIG. 12. Suturing and knot tying can be used with, or as an alternative to, the anchors 14, 16 to provide tissue fixation. The rectus muscle or fascia, the pubic symphysis, levator muscles, pubococcygeus muscle, puborectalis muscle, arcus tendineus fascia, puborectal ligament, or like available target locations can be the anchor site rather than the Cooper's ligament CL.

Physicians are able to use and adopt techniques more readily if the techniques are familiar to them. Also, supporting the perineal membrane PM with the rectus muscle/fascia can have the added benefit of pulling the perineal membrane PM upward when the rectus muscle moves outward due to abdominal pressure. This can serve to better treat men or women with severe incontinence. It has been shown that men require more tension on slings to provide effective treatment. These other anatomical structures may provide a beneficial anchoring force required to create continence in such circumstances.

The systems, their various components, structures, features, materials and methods of the present invention may have a number of suitable configurations as shown above. Various methods and tools for introducing, deploying, anchoring and manipulating implants or to treat incontinence and prolapse as disclosed in the previously-incorporated references are envisioned for use with the present invention as well.

A variety of materials may be used to form portions or components of the implants and devices, including Nitinol, polymers, elastomers, porous mesh, thermoplastic elastomers, metals, ceramics, springs, wires, plastic tubing, and the like. The systems, components and methods may have a number of suitable configurations known to one of ordinary skill in the art after reviewing the disclosure provided herein.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

What is claimed is:

1. A surgical implant system, comprising:
an implant device having a medial anchor engageable with a perineal membrane, a distal anchor engageable with an obturator muscle or membrane, and at least one suture length operatively connecting the medial anchor and the distal anchor; and
wherein the medial anchor is advanced via a percutaneous transobturator route, from the obturator foramen to the perineal membrane.

2. The system of claim 1, wherein the at least one suture length is a suture loop including a suture pair having first and second suture members.

3. The system of claim 2, wherein the distal anchor includes one or more apertures to receive the suture pair to provide selective tensioning.

4. The system of claim 1, wherein the medial anchor includes a plurality of extendable barbs defining an anchor array.

5. The system of claim 1, wherein the at least one suture length includes a knot member proximate a bottom surface of the distal anchor.

6. A surgical implant system, comprising:
an implant device having a medial anchor, a distal anchor, and at least one suture loop operatively connecting the medial anchor and the distal anchor, the at least one suture loop extending out from the distal anchor to facilitate selective tensioning of the implant; and
a delivery tool having a handle portion and a needle tip, the needle tip adapted to receive at least the medial anchor such that the medial anchor is introduced via percutaneous transobturator access, from an obturator tissue to a perineal membrane tissue site.

7. The system of claim 6, wherein the at least one suture loop is a suture pair having first and second suture members.

8. The system of claim 6, wherein the distal anchor includes one or more apertures to receive the at least one suture loop to facilitate the selective tensioning.

9. The system of claim 6, wherein the medial anchor includes a plurality of extendable barbs defining an anchor array.

10. The system of claim 6, further including a one-way stopper device.

11. The system of claim 10, wherein the one-way stopper device is provided along a portion of the at least one suture loop proximate the distal anchor.

12. The system of claim 6, wherein at least a portion of the at least one suture loop is quilled
